(19) United States
(12) Patent Application Publication
(10) Pub. No.: US 2023/0106359 A1
(43) Pub. Date: Apr. 6, 2023

(54) MULTI-USE ECG SYSTEM

(71) Applicant: BIOINTELLISENSE, INC., Golden, CO (US)

(72) Inventors: David Jonq Wang, Palo Alto, CA (US); James R. Mault, Evergreen, CO (US); Zongde Qiu, Cupertino, CA (US)

(21) Appl. No.: 17/485,315

(22) Filed: Sep. 24, 2021

(51) Int. Cl. A61B 5/00; A61B 5/366

(52) U.S. Cl. CPC A61B 5/6833; A61B 5/366

(57) ABSTRACT

In an example, an electrocardiogram (ECG) device includes a housing, an ECG sensor, and first and second electrodes. The ECG sensor is disposed in the housing. The first electrode is accessible from outside the housing and is electrically coupled to the ECG sensor. The second electrode is accessible from outside the housing and is electrically coupled to the ECG sensor. The housing and the first and second electrodes define an ECG device electromechanical interface that is complementary to a common patch electromechanical interface that is included in at least two different types of attachment patches.

### FIELD

The embodiments discussed herein are related to a multi-use electrocardiogram (ECG) system.

### BACKGROUND

Unless otherwise indicated herein, the materials described herein are not prior art to the claims in the present application and are not admitted to be prior art by inclusion in this section.

Cardiac sensors may be used for basic heart rate monitoring, arrhythmia detection and/or monitoring, and/or other uses. A distance between electrodes of such cardiac sensors may influence signal quality, which in turn may determine the suitability of detected signals for different uses.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In an example embodiment, an electrocardiogram (ECG) device includes a housing, an ECG sensor, and first and second electrodes. The ECG sensor is disposed in the housing. The first electrode is accessible from outside the housing and is electrically coupled to the ECG sensor. The second electrode is accessible from outside the housing and is electrically coupled to the ECG sensor. The housing and the first and second electrodes define an ECG device electromechanical interface that is complementary to a common patch electromechanical interface that is included in at least two different types of attachment patches.

In another example embodiment, a method includes forming an ECG device that includes a housing, an ECG sensor disposed in the housing, and first and second electrodes accessible from outside the housing and electrically coupled to the ECG sensor. The housing and the first and second electrodes define an ECG device electromechanical interface. The method also includes forming a first type of attachment patch that includes a common patch electromechanical interface that is complementary to the ECG device electromechanical interface and two first patch electrodes with a first spacing. The method also includes forming a second type of attachment patch that includes the common patch electromechanical interface that is complementary to the ECG device electromechanical interface and two second patch electrodes with a second spacing that is greater than the first spacing.

In another example embodiment, an ECG system includes an ECG device, a first type of attachment patch, and a second type of attachment patch. The ECG device includes a housing, an ECG sensor disposed in the housing, and first and second electrodes accessible from outside the housing and electrically coupled to the ECG sensor. The housing and the first and second electrodes define an ECG device electromechanical interface. The first type of attachment patch includes a common patch electromechanical interface that is complementary to the ECG device electromechanical interface and two first patch electrodes with a first spacing. The second type of attachment patch includes the common patch electromechanical interface that is complementary to the ECG device electromechanical interface and two second patch electrodes with a second spacing that is greater than the first spacing.

In another example embodiment, an ECG system includes an ECG device and a non-arrythmia attachment patch. The ECG device includes a housing, an ECG sensor disposed in the housing, and first and second electrodes accessible from outside the housing and electrically coupled to the ECG sensor. The housing and the first and second electrodes define an ECG device electromechanical interface. The non-arrythmia attachment patch includes a common patch electromechanical interface that is complementary to the ECG device electromechanical interface and two patch electrodes with a predetermined spacing. The non-arrythmia attachment patch is configured to be electrically coupled to the ECG device through the common patch electromechanical interface and the ECG device electromechanical interface and to skin of a subject through the two patch electrodes. The non-arrythmia attachment patch is configured to direct electrical signals from locations of the subject at which the two patch electrodes are positioned when the non-arrythmia attachment patch is coupled to the skin of the subject and spaced apart by the predetermined spacing to, respectively, the first and second electrodes of the ECG device.

In another example embodiment, an ECG system includes an ECG device and an arrhythmia attachment patch. The ECG device includes a housing, an ECG sensor disposed in the housing, and first and second electrodes accessible from outside the housing and electrically coupled to the ECG sensor. The housing and the first and second electrodes define an ECG device electromechanical interface. The arrythmia attachment patch includes a common patch electromechanical interface that is complementary to the ECG device electromechanical interface and two patch electrodes with a predetermined spacing. The arrythmia attachment patch is configured to be electrically coupled to the ECG device through the common patch electromechanical interface and the ECG device electromechanical interface and to skin of a subject through the two patch electrodes. The arrythmia attachment patch is configured to direct electrical signals from locations of the subject at which the two patch electrodes are positioned when the arrythmia attachment patch is coupled to the skin of the subject and spaced apart by the predetermined spacing to, respectively, the first and second electrodes of the ECG device.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a graph including an example trace representing a normal heart rhythm;

FIGS. 2A and 2B illustrate an example operating environment;

FIGS. 3A-3C illustrate an example ECG device that may be implemented in ECG systems of FIGS. 2A-2B;

FIGS. 4A-4C include an overhead view, a bottom view, and a cross-sectional view of a non-arrythmia attachment patch that may be implemented in the ECG systems of FIGS. 2A-2B;

FIG. 5 includes a cross-sectional view of an ECG system that includes the ECG device of FIGS. 3A-3C and the non-arrythmia attachment patch of FIGS. 4A-4C;

FIGS. 6A-6C include an overhead view, a bottom view, and a cross-sectional view of an arrhythmia attachment patch that may be implemented in the ECG systems of FIGS. 2A-2B;

FIG. 7 includes a cross-sectional view of an ECG system that includes the ECG device of FIGS. 3A-3C and the arrhythmia attachment patch of FIGS. 6A-6C;

FIGS. 8A and 8B are bottom views of other attachment patches that may be implemented in ECG systems;

FIG. 9 is a flowchart of a manufacturing method; and

FIG. 10 is a block diagram illustrating an example computing device,

all arranged in accordance with at least one embodiment described herein.

### DETAILED DESCRIPTION OF SOME EXAMPLE EMBODIMENTS

Some embodiments herein relate to multi-use ECG systems that include an ECG device and at least one of multiple different types of attachment patches, each of the different types of attachment patches having a different use. All of the different types of attachment patches may include a common patch electromechanical interface that may be configured to electromechanically couple the corresponding type of attachment patch to the ECG device. Accordingly, a single type of ECG device may be implemented with any one of multiple different types of attachment patches.

Reference will now be made to the drawings to describe various aspects of example embodiments of the invention. It is to be understood that the drawings are diagrammatic and schematic representations of such example embodiments, and are not limiting of the present invention, nor are they necessarily drawn to scale.

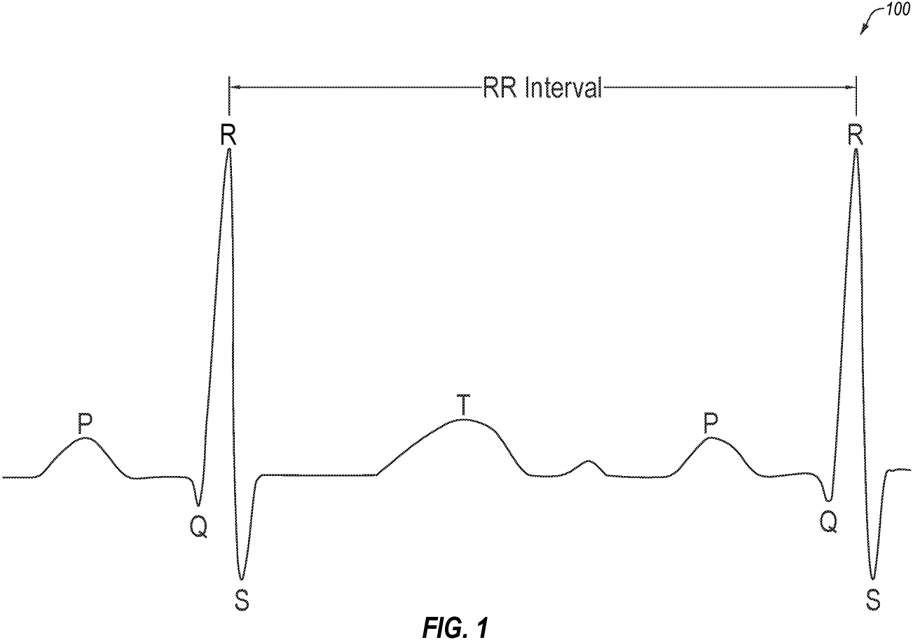

FIG. 1 is a graph including an example trace 100 representing a normal heart rhythm, arranged in accordance with at least one embodiment described herein. A cardiac sensor such as an electrocardiography (ECG or EKG) device may be configured to generate such a trace by detecting electrical signals generated by the sinoatrial (SA) node of the heart, which electrical signals control the heart’s rhythm.

The trace 100 includes various waves or portions labeled P, Q, R, S and T, which are sometimes grouped together and described as a complex, such as the QRS complex. In a normal heart rhythm, the SA node generates an electrical impulse which travels through the right and left atria. The P wave represents the electricity flowing through the atria. The QRS complex represents the flow through the ventricles as they contract to push the blood out from the heart. The T wave represents repolarization or the electrical resetting of the heart for the next beat. The next heart beat cycle begins at the next P wave. In a normal heart rhythm, the heart beat cycles are usually regular, meaning the portion of the trace 100 for one heart beat cycle is substantially similar to the portion of the trace 100 for the next heart beat cycle.

Heart rate is often described in terms of beats per minute. One method of calculating heart rate involves determining the time between successive R waves, known as the RR interval (RRI). Heart rate in terms of beats per minute is inversely proportional to the RRI and may be calculated from the RRI. The RRI may be determined from a trace generated by an ECG device, such as the trace 100 of FIG. 1, or more generally from a data signal indicating a heart rate of a subject over time, which data signal may be generated by any suitable cardiac sensor. An instantaneous heart rate may be obtained from a single complete heart beat cycle, e.g., from one R wave to the next, or averaged over multiple heart beat cycles.

Cardiac sensors may be used for basic heart rate monitoring, arrhythmia detection and/or monitoring, and/or other uses depending on a number of signal collection nodes, their relative arrangements, and/or the spacing therebetween. Basic heart rate monitoring typically primarily or exclusively detects the RRI. Given the significant peak magnitude of the R wave, basic heart rate monitoring may be implemented with relatively low signal quality. In comparison, arrhythmia detection and/or monitoring may involve analysis of the PQRS waveform and may therefore benefit from and/or require higher signal quality than basic heart rate monitoring. Signal quality may be affected by, among potentially other factors, a distance between signal collection nodes of the cardiac sensor on a subject.

Some embodiments herein relate to cardiac sensors that may be combined in a system with one of at least two or more application-specific attachment patches for one of at least two specific applications. More particularly, some embodiments herein may include an ECG system made up of an ECG device that includes an ECG sensor electrically coupled to an ECG device electromechanical interface of the ECG device and at least two different types of attachment patches. Each of the attachment patches may have a common patch electromechanical interface configured to cooperate with the ECG device electromechanical interface to electromechanically couple the ECG device to the corresponding attachment patch. Moreover, different types of attachment patches may have different arrangements of electrodes coupled to the corresponding common patch electromechanical interface (and therethrough to the ECG sensor) for different specific applications. For example, one type of attachment patch which may be specifically used for fitness applications or remote cardiac monitoring and which is primarily concerned with basic heart rate monitoring, referred to herein as a non-arrythmia monitoring type attachment patch or simply non-arrythmia attachment patch, may have two electrodes with a relatively narrow spacing. Another type of attachment patch which may be specifically used for arrhythmia detection and/or monitoring applications and which is primarily concerned with detecting and/or monitoring arrhythmia, referred to herein as an arrhythmia type attachment patch or simply arrhythmia attachment patch, may have two electrodes with a relatively wider spacing for improved signal quality.

Embodiments herein may enable a single sensor platform, e.g., any instance of the ECG device, to be used with any one of multiple different types of attachment patches for any one of multiple different specific applications. The use of a single or common sensor platform for multiple attachment patches and/or applications may reduce development and/or manufacturing costs compared to using different sensor platforms for different attachment patches and/or applications.

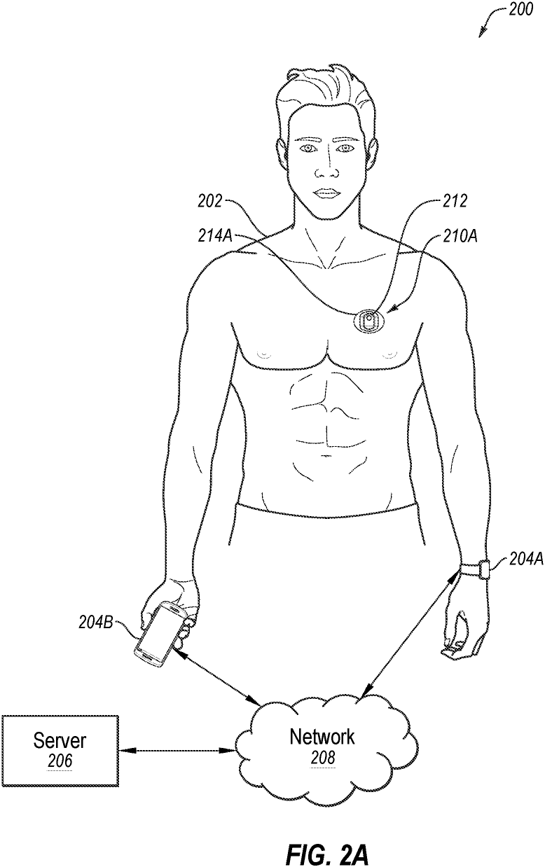

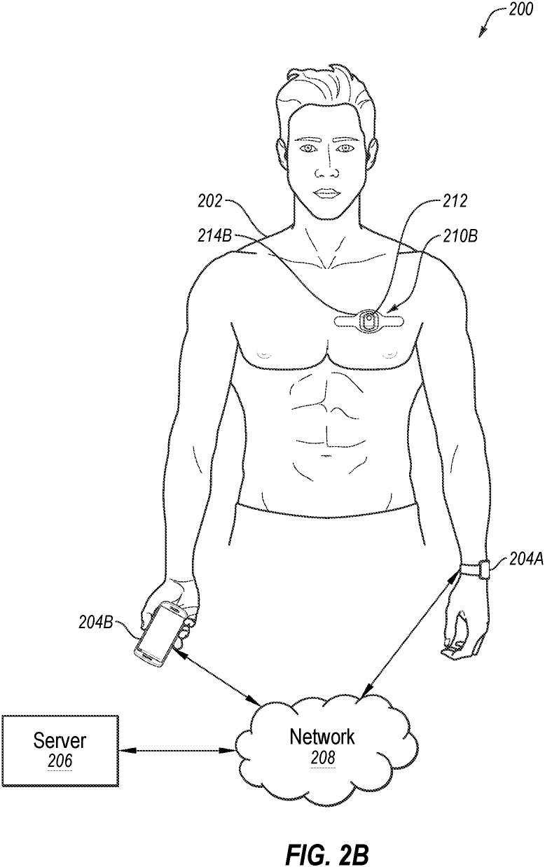

FIGS. 2A and 2B illustrate an example operating environment 200 (hereinafter “environment 200”), arranged in accordance with at least one embodiment described herein. The environment 200 includes a subject 202 and one or more personal electronic devices 204A, 204B (hereinafter collectively “personal electronic devices 204” or generically “personal electronic device 204”). The environment 200 may additionally include a server 206 and a network 208.

FIG. 2A depicts an ECG system 210A in the environment 200 while FIG. 2B depicts an ECG system 210B in the environment 200. The ECG systems 210A, 210B may be collectively referred to herein as “ECG systems 210” or generically as “ECG system 210”.

Each of the ECG systems 210 may include an ECG device 212 and an attachment patch 214A or 214B (hereinafter collectively “attachment patches 214” or generically “attachment patch 214”). The ECG devices 212 in the ECG systems 210 may be identical, e.g., different instances of the same ECG sensor platform, with the same dimensions (within tolerances), components, etc. The attachment patches 214 may be different types of attachment patches. For example, the attachment patch 214A of FIG. 2A may be a first type of attachment patch, such as a non-arrythmia attachment patch, while the attachment patch 214B of FIG. 2B may be a second type of attachment patch, such as an arrhythmia attachment patch. In some embodiments, non-arrythmia attachment patches may be configured to cooperate with the ECG device 212 to measure timing between successive R-R waveforms of the subject, or RRI. Alternatively or additionally, arrhythmia attachment patches may be configured to cooperate with the ECG device 212 to measure a PQRS waveform of the subject. Using the same ECG sensor platform as described herein with different types of attachment patches and/or for different specific applications may reduce development and/or manufacturing costs compared to using different ECG sensor platforms with different types of attachment patches and/or for different specific applications. The ECG systems 210 may generate ECG measurement data alone, or additionally may generate one or more other types of measurement data, such as temperature measurement data, movement measurement data, respiratory measurement data, or the like, collectively or generically hereinafter referred to as “measurement data”. The ECG systems 210 may provide the measurement data to the personal electronic devices 204 and/or the server 206.

The personal electronic devices 204 may each include a desktop computer, a laptop computer, a tablet computer, a smartphone, a wearable electronic device (e.g., smart watch, activity tracker, headphones, ear buds, etc.), or other personal electronic device. In the illustrated example, the personal electronic device 204A may include a smart watch and the personal electronic device 204B may include a smartphone. In some embodiments, the personal electronic devices 204 may collect measurement data from the ECG systems 210 for use and/or analysis on the personal electronic devices 204.

Alternatively or additionally, the measurement data generated by the ECG system 210 and/or data derived therefrom may be uploaded, e.g., periodically, by the ECG system 210 to the remote server 206. In some embodiments, one or more of the personal electronic devices 204 or another device may act as a hub that collects measurement data or data derived therefrom from the ECG system 210 and/or other personal electronic devices 204 and uploads the measurement data or data derived therefrom to the server 206. For example, the hub may collect data over a local communication scheme (WI-FI, BLUETOOTH, near-field communications (NFC), etc.) and may transmit the data to the server 206. In some embodiments, the hub may act to collect the data and periodically provide the data to the server 206, such as once per week. An example hub and associated methods and devices are disclosed in U.S. Pat. No. 10,743,091, which is incorporated herein by reference.

The server 206 may include a collection of computing resources available in the cloud and/or a discrete server computer. The server 206 may be configured to receive measurement data and/or data derived from measurement data from one or more of the personal electronic devices 204 and/or from the ECG system 210. Alternatively or additionally, the server 206 may be configured to receive from the ECG system 210 (e.g., directly or indirectly via a hub device) relatively small portions of the measurement data, or even larger portions or all of the measurement data. The server 206 may use and/or analyze the data, e.g., to detect and/or monitor the heart rate of the subject 202, arrhythmia of the subject 202, or the like. Alternatively or additionally, the server 206 may store the measurement data in an account of the subject 202 and make the measurement data or data derived therefrom available to the subject 202, a healthcare provider, or other individuals, e.g., as authorized by the subject 202 e.g., via an online portal.

The network 208 may include one or more wide area networks (WANs) and/or local area networks (LANs) that enable the personal electronic devices 204, the server 206, and/or the ECG system 210 to communicate with each other. In some embodiments, the network 208 includes the Internet, including a global internetwork formed by logical and physical connections between multiple WANs and/or LANs. Alternately or additionally, the network 208 may include one or more cellular radio frequency (RF) networks and/or one or more wired and/or wireless networks such as 802.xx networks, BLUETOOTH access points, wireless access points, IP-based networks, or other suitable networks. The network 208 may also include servers that enable one type of network to interface with another type of network.

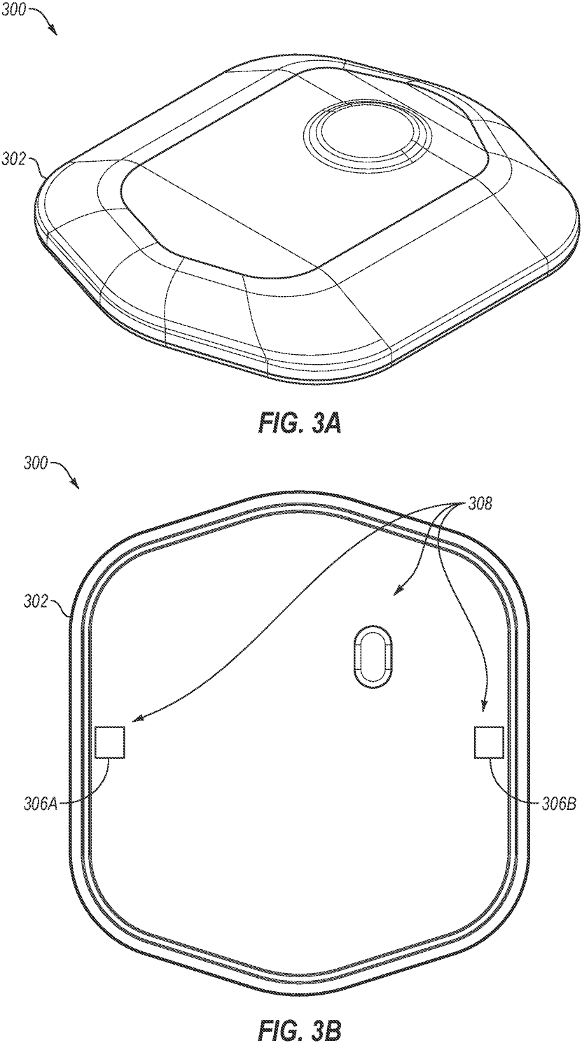

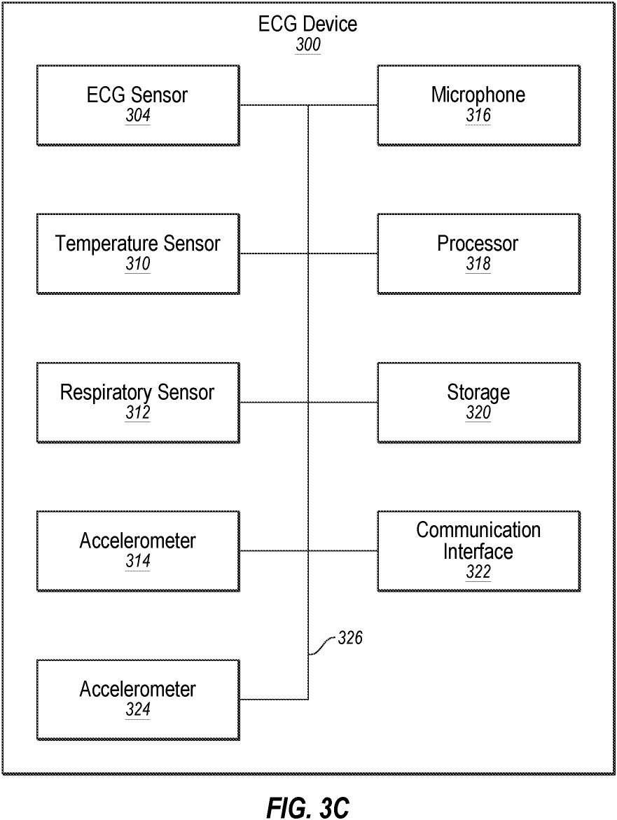

FIGS. 3A-3C illustrate an example ECG device 300 that may be implemented in the ECG systems 210 of FIGS. 2A-2B, arranged in accordance with at least one embodiment described herein. FIG. 3A includes a top front perspective view of the ECG device 300, FIG. 3B includes a bottom view of the ECG device 300, and FIG. 3C includes a block diagram of the ECG device 300. The ECG device 300 may include, be included in, or correspond to the ECG device 212 of FIGS. 2A-2B and/or other ECG devices described herein.

In general, the ECG device 300 may include a housing 302 (FIGS. 3A-3B) and an ECG sensor 304 (FIG. 3C) disposed in the housing 302. In general, the ECG sensor 304 may be configured to detect electrical signals generated by the SA node of the heart of a subject, such as of the subject 202 and to generate ECG measurement data that represents or corresponds to the detected electrical signals. The ECG sensor 304, the ECG device 300, a processor of the ECG device 300, and/or other device or system (such as one or more of the personal electronic devices 204 and/or the server 206) may determine, based on the ECG measurement data, the RRI of the subject, the heart rate of the subject, the PQRS complex of the subject, or other parameters of the subject as instantaneous measurements, average measurements, time series of instantaneous and/or average measurements, or the like.

The ECG device 300 may further include first and second electrodes 306A, 306B (FIGS. 3A-3B) (hereinafter collectively “electrodes 306”) accessible from outside the housing 302 and electrically coupled to the ECG sensor 304. For example, the electrodes 306 may protrude from a bottom of the housing 302 and may be electrically coupled to the ECG sensor 304 through one or more wires, printed circuit board (PCB) traces, bond pads, or the like. While only two electrodes 306 are illustrated in FIG. 3B, more generally the ECG device 300 may include two or more electrodes 306 electrically coupled to the ECG sensor 304 and accessible from outside the housing 302. When the ECG device 300 includes more than two electrodes 306, the ECG device 300 may be used for, e.g., multi-lead ECG applications.

The housing 302 and the electrodes 306 define an ECG device electromechanical interface 308 (FIG. 3B). The ECG device electromechanical interface 308 in some embodiments may include a bottom surface of the housing 302 and the electrodes 307. The ECG device electromechanical interface 308 may be configured to electromechanically couple the ECG device 300 to attachment patches, such as the attachment patches 214 of FIGS. 2A-2B. For example, the bottom surface of the housing 302 together with an adhesive may mechanically couple the ECG device 300 to an attachment patch and the electrodes 306 may electrically couple the ECG device 300 to the attachment patch.

As illustrated in FIG. 3C, the ECG device 300 may further include one or more of a temperature sensor 310, a respiratory sensor 312, an accelerometer 314, a microphone 316, a processor 318, storage 320, a communication interface 322, a battery 324, a communication bus 326, and/or other sensors, components, or devices.

The temperature sensor 310 may be configured to detect temperatures associated with a subject, such as skin temperature and/or core body temperature and to generate temperature measurement data that represents or corresponds to the detected temperature(s).

The respiratory sensor 312 may be configured to detect respiration of the subject and to generate respiratory measurement data that represents or corresponds to the detected respiration.

The accelerometer 314 may be configured to detect movement of the subject and to generate movement measurement data that represents or corresponds to the detected movement. In some embodiments, the accelerometer 314 may be used specifically to measure acceleration of at least a portion of the subject, such as the chest of the subject, based on the ECG device 300 being adhered to the portion of the subject.

The microphone 316 may be used to record sound and may be oriented to face the skin of the subject. While the term microphone is used, it will be appreciated that term includes any type of acoustic sensor that may be configured to detect sound waves and convert them into a readable signal such as an electronic signal. For example, a piezoelectric transducer, a condenser microphone, a moving-coil microphone, a fiber optic microphone, a MicroElectrical-Mechanical System (MEMS) microphone, etc. or any other transducer may be used to implement the microphone 316.

Although not illustrated in FIG. 3C, the ECG device 300 may include one or more other sensors, such as a gyrometer sensor, a blood pressure sensor, an optical spectrometer sensor, an electro-chemical sensor, an oxygen saturation sensor, a photoplethysmography (PPG) sensor, an electrodermal activity (EDA) sensor, a volatile organic compound (VOC) sensor, an optical sensor, a spectrometer, or any combination thereof. A gyrometer sensor may be used to measure angular velocity of at least a portion of the subject, such as the chest of subject. An oxygen saturation sensor may be used to record blood oxygenation of the subject. A PPG sensor may be used to record blood flow of the subject. An EDA sensor may be used to measure EDA of the skin of the subject. A volatile organic compound (VOC) detector may be used to detect various organic molecules that may be coming off of the subject or that may be in the subject’s sweat. An optical sensor may be used to monitor or detect changes in color, such as changes in skin coloration of the subject. A spectrometer may measure electromagnetic (EM) radiation and may be configured to detect variations in reflected EM radiation. For example, such a sensor may detect changes in color in a molecule exposed to multi-spectral light (e.g., white light), and/or may detect other changes in reflected EM radiation outside of the visible spectrum (e.g., interaction with ultra-violet rays, etc.).

The processor 318 may include any device or component configured to monitor and/or control operation of the ECG device 300. For example, the processor 318 may retrieve instructions from the storage 320 and execute those instructions. As another example, the processor 318 may read the signals and/or measurement data generated by sensors (e.g., the ECG sensor 304, the temperature sensor 310, the respiratory sensor 312, the accelerometer 314, the microphone 316, and/or other sensors) and may store the readings in the storage 320 or instruct the communication interface 322 to send the readings to another electronic device, such as the server 206 of FIGS. 2A-2B. In some embodiments, the processor 318 may include an arithmetic logic unit, a microprocessor, a general-purpose controller, or some other processor or array of processors, to perform or control performance of operations as described herein. The processor 318 may be configured to process data signals and may include various computing architectures including a complex instruction set computer (CISC) architecture, a reduced instruction set computer (RISC) architecture, or an architecture implementing a combination of instruction sets. Although illustrated as a single processor 318, multiple processor devices may be included and other processors and physical configurations may be possible. The processor 318 may be configured to process any suitable number format including, but not limited to two’s compliment numbers, integers, fixed binary point numbers, and/or floating point numbers, etc. all of which may be signed or unsigned. In some embodiments, the processor 318 may perform processing on the readings from the sensors prior to storing and/or communicating the readings. For example, raw analog data signals generated by the ECG sensor 304, the temperature sensor 310, the respiratory sensor 312, the accelerometer 314, the microphone 316, and/or other sensors of the ECG device 300 may be downsampled, may be converted to digital data signals, and/or may be processed in some other manner.

The storage 320 may include non-transitory computer-readable storage media or one or more non-transitory computer-readable storage mediums for carrying or having computer-executable instructions or data structures stored thereon. Such non-transitory computer-readable storage media may be any available non-transitory media that may be accessed by a general-purpose or special-purpose computer, such as the processor 318. By way of example such non-transitory computer-readable storage media may include Random Access Memory (RAM), Read-Only Memory (ROM), Electrically Erasable Programmable Read-Only Memory (EEPROM), flash memory devices (e.g., solid state memory devices), or any other non-transitory storage medium which may be used to carry or store desired program code in the form of computer-executable instructions or data structures and which may be accessed by a general-purpose or special-purpose computer. In some embodiments, the storage 320 may alternatively or additionally include volatile memory, such as a dynamic random access memory (DRAM) device, a static random access memory (SRAM) device, or the like. Combinations of the above may also be included within the scope of non-transitory computer-readable storage media. Computer-executable instructions may include, for example, instructions and data that when executed by the processor 318 cause the processor 318 to perform or control performance of a certain operation or group of operations. In some embodiments, the storage 320 may store the data signals, e.g., measurement data, generated by the ECG sensor 304, the temperature sensor 310, the respiratory sensor 312, the accelerometer 314, the microphone 316, and/or other sensors of the ECG device 300 and/or data derived therefrom.

The communication interface 322 may include any device or component that facilitates communication with a remote device, such as any of the personal electronic devices 204 of the subject 202, the server 206, or any other electronic device. For example, the communication interface 322 may include an RF antenna, an infrared (IR) receiver, a WI-FI chip, a BLUETOOTH chip, a cellular chip, a near-field communication (NFC) chip, or any other communication interface.

The battery 324 may include any device or component configured to provide power to the ECG device 300 and/or the components thereof. For example, the battery 324 may include a rechargeable battery, a disposable battery, etc. In some embodiments, the ECG device 300 may include circuitry, electrical wires, etc. to provide power from the battery 324 to the other components of the ECG device 300. In some embodiments, the battery 324 may include sufficient capacity such that the ECG device 300 may operate for days, weeks, or months without having the battery changed or recharged. For example, the ECG device 300 may be configured to operate for at least two months without having the battery 324 charged or replaced.

The communication bus 326 may include any connections, lines, wires, or other components facilitating communication between the various components of the ECG device 300. The communication bus 326 may include one or more hardware components and may communicate using one or more protocols. Additionally or alternatively, the communication bus 326 may include wire connections between the components. In some embodiments, the ECG device 300 may operate in a similar or comparable manner to the embodiments described in U.S. Application No. 17/349,166 filed on Jun. 16, 2021 and/or U.S. Pub. No. 2020/0069281, both of which are hereby incorporated by reference.

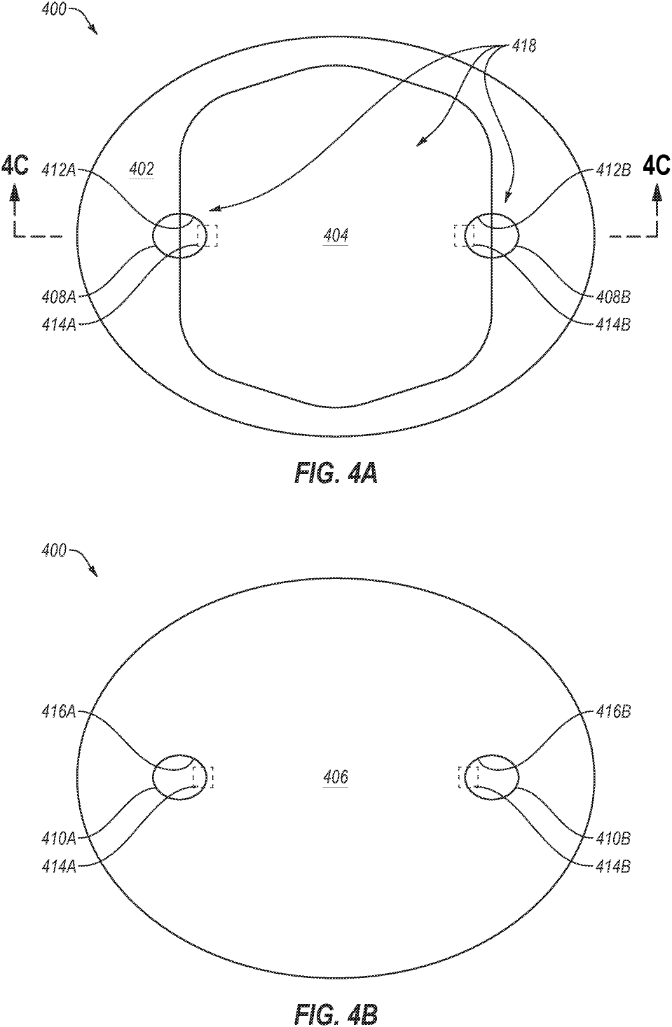

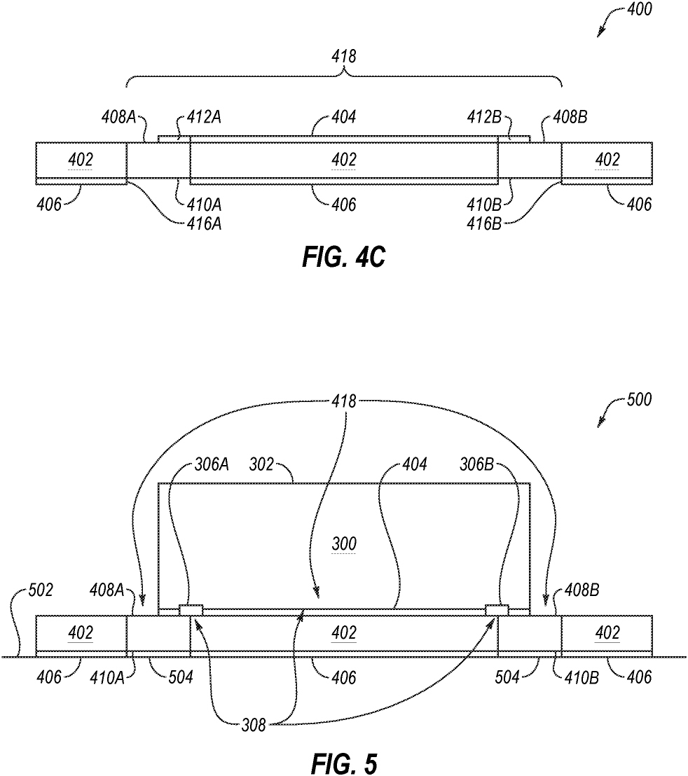

FIGS. 4A-4C include an overhead view, a bottom view, and a cross-sectional view of a non-arrythmia attachment patch 400 that may be implemented in the ECG systems 210 of FIGS. 2A-2B, arranged in accordance with at least one embodiment described herein. The cross-sectional view of FIG. 4C is taken along cutting plane 4C-4C in FIG. 4A. The non-arrythmia attachment patch 400 may include, be included in, or correspond to the attachment patch 214A of FIG. 2A and/or other attachment patches described herein. In general, the non-arrythmia attachment patch 400 may be configured to couple an ECG device, such as the ECG device 300 of FIGS. 3A-3C, to a subject, such as the subject 202 of FIG. 2A. For simplicity in the discussion that follows, the non-arrythmia attachment patch 400 will be described in the context of coupling the ECG device 300 to the subject 202.

In some embodiments, the non-arrythmia attachment patch 400 may include a backing material or substrate 402 (FIGS. 4A and 4C) with a first adhesive layer 404 formed or deposited on a device-facing side thereof and a second adhesive layer 406 formed or deposited on an opposite or skin-facing side thereof. The backing material 402 may include any suitable backing material. The first and second adhesive layers 404, 406 may include any suitable adhesive. The non-arrythmia attachment patch 400 may additionally include one or more electrode contacts 408A, 408B, illustrated in FIG. 4A as first and second electrode contacts 408A, 408B (hereinafter collectively “electrode contacts 408” or generically “electrode contact 408”) and one or more patch electrodes 410A, 410B, illustrated in FIG. 4B as first and second patch electrodes 410A, 410B (hereinafter collectively “patch electrodes 410” or generically “patch electrode 410”).

The first adhesive layer 404 may be formed or deposited on all or a portion of the device-facing side of the backing material 402. In the illustrated embodiment of FIG. 4A, the first adhesive layer 404 is formed or deposited on a portion of the device-facing side of the backing material 402 in a confined area that may correspond to an area of the bottom surface of the ECG device 300 of FIGS. 3A-3C. For example, the first adhesive layer 404 may have the same or similar shape and/or dimensions as the bottom surface of the housing 302 of the ECG device 300. Alternatively or additionally, the first adhesive layer 404 may have a different shape and/or different dimensions (e.g., larger, smaller, and/or up to the entire device-facing side of the backing material 402). Forming or depositing the first adhesive layer 404 on the device-facing side of the backing material 402 in the same or similar shape and dimensions as the bottom surface of the housing 302 may facilitate visual alignment and attachment of the ECG device 300 to the non-arrythmia attachment patch 400 by the subject 202, a healthcare worker, or other individual.

The first adhesive layer 404 may include cutouts 412A, 412B (hereinafter collectively “cutouts 412” or generically “cutout 412”) around the electrode contacts 408 to reduce or eliminate interference of the first adhesive layer 404 with an electrical connection between the electrode contacts 408 of the non-arrythmia attachment patch 400 and the electrodes 306 of the ECG device 300. Approximate locations of the electrodes 306 of the ECG device 300 when coupled to the device-facing side (FIG. 4A) of the non-arrythmia attachment patch 400 are designated in FIGS. 4A-4B by dashed boxes 414A, 414B (hereinafter “ECG device electrode locations 414”).

The second adhesive layer 406 may be formed or deposited on all or a portion of the skin-facing side of the backing material 402. In the illustrated embodiment of FIG. 4B, the second adhesive layer 406 is formed or deposited on substantially all of the skin-facing side of the backing material 402. In other embodiments, the second adhesive layer 406 may be formed or deposited in a confined area or areas of the skin-facing side of the backing material 402. FIG. 4B further illustrates cutouts 416A, 416B (hereinafter collectively “cutouts 416”) that may be included in the second adhesive layer 406 around the patch electrodes 410. The cutouts 416 may reduce or eliminate interference of the second adhesive layer 406 with an electrical connection between the patch electrodes 410 of the non-arrythmia attachment patch 400 and skin of the subject 202.

The electrode contacts 408 may include metal, metallization, electrically conductive ink, or other electrically conductive material(s) or structure(s) formed or deposited in or on the backing material 402. The electrode contacts 408 may be exposed or accessible at the device-facing side of the backing material 402. In addition, the electrode contacts 408 may be disposed at locations of the backing material 402 that at least partially align with the ECG device electrode locations 414. As such, the electrode contacts 408 may electrically couple the non-arrythmia attachment patch 400 to the ECG device 300, and specifically to the electrodes 306 of the ECG device 300, when the non-arrythmia attachment patch 400 is properly aligned with and coupled to the ECG device 300. In particular, when the ECG device 300 is coupled to the non-arrythmia attachment patch 400 with the ECG device 300 aligned to the non-arrythmia attachment patch 400 such that the electrodes 306 of the ECG device 300 are aligned to the ECG device electrode locations 414, the first electrode contact 408A may be electrically coupled to the first electrode 306A of the ECG device 300 and the second electrode contact 408B may be electrically coupled to the second electrode 306B of the ECG device 300.

A portion of the device-facing side of the backing material 402 to which the ECG device 300 is coupled, generally corresponding to the first adhesive layer 404 in this example, together with the electrode contacts 408 and/or the first adhesive layer 404, may define a common patch electromechanical interface 418. The common patch electromechanical interface 418 may include the portion of the device-facing side of the backing material 402, the electrode contacts 408 positioned to align with and couple to the electrodes 306 of the ECG device 300, and/or the first adhesive layer 404. The common patch electromechanical interface 418 may be configured to electromechanically couple the non-arrythmia attachment patch 400 to ECG devices, such as the ECG device 300. For example, the portion of the device-facing side of the backing material 402, together with the first adhesive layer 404, may mechanically couple the non-arrythmia attachment patch 400 to the ECG device 300 and the electrode contacts 408 may electrically couple the non-arrythmia attachment patch 400 to the electrodes 306 of the ECG device 300.

The patch electrodes 410 may include metal, metallization, electrically conductive ink, or other electrically conductive material(s) or structure(s) formed or deposited in or on the backing material 402. The patch electrodes 410 may be exposed or accessible at the skin-facing side of the backing material 402. In addition, the patch electrodes 410 may have a spacing according to a desired use of the non-arrythmia attachment patch 400. For example, in some embodiments, the patch electrodes 410 may be space about 35 millimeters (mm) apart, or other distance. The spacing of the patch electrodes 410 may refer to a center-to-center spacing of the patch electrodes 410. Moreover, when the term “about” is applied to a measurement or parameter, it may include the stated value for the measurement or parameter plus or minus 15%.

The patch electrodes 410 may be electrically coupled to the electrode contacts 408. In the illustrated embodiment, the metal, metallization, electrically conductive ink, or other electrically conductive material(s) or structure(s) of the patch electrodes 410 and the metal, metallization, electrically conductive ink, or other electrically conductive material(s) or structure(s) of the electrode contacts 408 are one and the same. As such, in this embodiment, the metal, metallization, electrically conductive ink, or other electrically conductive material(s) or structure(s) extends through the backing material 402 to expose the electrode contacts 408 at the device-facing side of the backing material 402 and the patch electrodes 410 at the skin-facing side of the backing material 402. In other embodiments, the patch electrodes 410 may be laterally spaced apart from the electrode contacts 408 and may be electrically coupled to the electrode contacts 408 through one or more electrically conductive structures, such as through one or more electrical traces, one or more wires, one or more nanowires, or one or more electrically conductive ink structures. The electrical connections between the patch electrodes 410 and the electrode contacts 408 allows the non-arrythmia attachment patch 400 to direct electrical signals from locations of the subject at which the patch electrodes

FIG. 5 includes a cross-sectional view of an ECG system 500 that includes the ECG device 300 of FIGS. 3A-3C and the non-arrythmia attachment patch 400 of FIGS. 4A-4C, arranged in accordance with at least one embodiment described herein. The cross-sectional view of FIG. 5 is taken from the same direction as the cross-sectional view of FIG. 4C with the addition of the ECG device 300 electromechanically coupled to the non-arrythmia attachment patch 400.

As illustrated in FIG. 5, the ECG device electromechanical interface 308 and the common patch electromechanical interface 418 cooperate to electromechanically couple the ECG device 300 to the non-arrythmia attachment patch 400. In particular, the electrodes 306 of the ECG device 300 and the electrode contacts 408 of the non-arrythmia attachment patch 400 cooperate to electrically couple the ECG device 300 to the non-arrythmia attachment patch 400 while the bottom surface of the housing 302 of the ECG device 300, the portion of the device-facing side of the backing material 402, and the first adhesive layer 404 cooperate to mechanically couple the ECG device 300 to the non-arrythmia attachment patch 400.

FIG. 5 further illustrates the ECG system 500 coupled to skin 502 of a subject, such as the subject 202 of FIG. 2A. In particular, the second adhesive layer 406 may mechanically couple the ECG system 500 to the skin 502. In some embodiments, a hydrogel 504 or other electrically conductive substance may be placed on the patch electrodes 410, e.g., within the cutouts 416 (FIGS. 4B-4C), before the ECG system 500 is coupled to the skin 502 to electrically couple the skin 502 to the patch electrodes 410, and more generally to the ECG system 500, when the ECG system 500 is mechanically coupled to the skin 502.

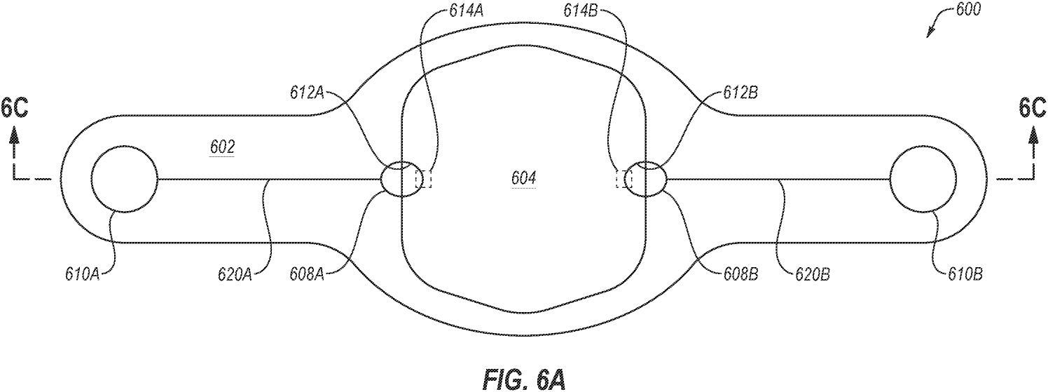

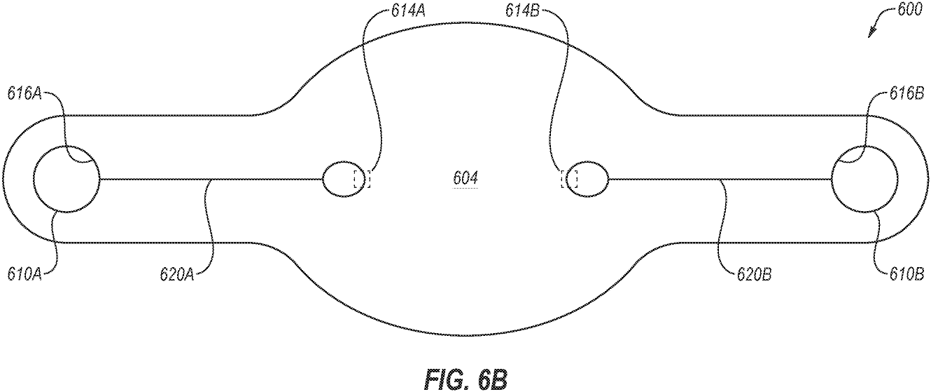

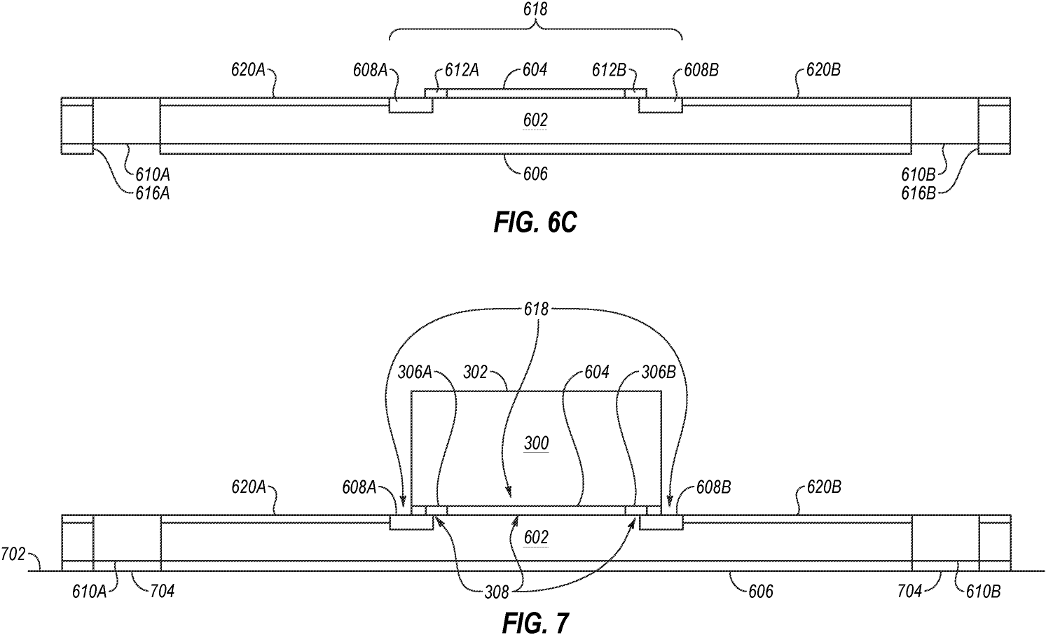

FIGS. 6A-6C include an overhead view, a bottom view, and a cross-sectional view of an arrhythmia attachment patch 600 that may be implemented in the ECG systems 210 of FIGS. 2A-2B, arranged in accordance with at least one embodiment described herein. The cross-sectional view of FIG. 6C is taken along cutting plane 6C-6C in FIG. 6A. The arrhythmia attachment patch 600 may include, be included in, or correspond to the attachment patch 214B of FIG. 2B and/or other attachment patches described herein. In general, the arrhythmia attachment patch 600 may be configured to couple an ECG device, such as the ECG device 300 of FIGS. 3A-3C, to a subject, such as the subject 202 of FIG. 2B. For simplicity in the discussion that follows, the arrhythmia attachment patch 600 will be described in the context of coupling the ECG device 300 to the subject 202.

In some embodiments, the arrhythmia attachment patch 600 may include a backing material or substrate 602 (FIGS. 6A and 6C) with a first adhesive layer 604 formed or deposited on a device-facing side thereof and a second adhesive layer 606 formed or deposited on an opposite or skin-facing side thereof. The backing material 602 may include any suitable backing material. The first and second adhesive layers 604, 606 may include any suitable adhesive. The arrhythmia attachment patch 600 may additionally include one or more electrode contacts 608A, 608B, illustrated in FIG. 6A as first and second electrode contacts 608A, 608B (hereinafter collectively “electrode contacts 608” or generically “electrode contact 608”) and one or more patch electrodes 610A, 610B, illustrated in FIG. 6B as first and second patch electrodes 610A, 610B (hereinafter collectively “patch electrodes 610” or generically “patch electrode 610”).

The first adhesive layer 604 may be formed or deposited on all or a portion of the device-facing side of the backing material 602. In the illustrated embodiment of FIG. 6A, the first adhesive layer 604 is formed or deposited on a portion of the device-facing side of the backing material 602 in a confined area that may correspond to an area of the bottom surface of the ECG device 300 of FIGS. 3A-3C. For example, the first adhesive layer 604 may have the same or similar shape and/or dimensions as the bottom surface of the housing 302 of the ECG device 300. Alternatively or additionally, the first adhesive layer 604 may have a different shape and/or different dimensions (e.g., larger, smaller, and/or up to the entire device-facing side of the backing material 602). Forming or depositing the first adhesive layer 604 on the device-facing side of the backing material 602 in the same or similar shape and dimensions as the bottom surface of the housing 302 may facilitate visual alignment and attachment of the ECG device 300 to the arrhythmia attachment patch 600 by the subject 202, a healthcare worker, or other individual.

The first adhesive layer 604 may include cutouts 612A, 612B (hereinafter collectively “cutouts 612” or generically “cutout 612”) around the electrode contacts 608 to reduce or eliminate interference of the first adhesive layer 604 with an electrical connection between the electrode contacts 608 of the arrhythmia attachment patch 600 and the electrodes 306 of the ECG device 300. Approximate locations of the electrodes 306 of the ECG device 300 when coupled to the device-facing side (FIG. 6A) of the arrhythmia attachment patch 600 are designated in FIGS. 6A-6B by dashed boxes 614A, 614B (hereinafter “ECG device electrode locations 614”).

The second adhesive layer 606 may be formed or deposited on all or a portion of the skin-facing side of the backing material 602. In the illustrated embodiment of FIG. 6B, the second adhesive layer 606 is formed or deposited on substantially all of the skin-facing side of the backing material 602. In other embodiments, the second adhesive layer 606 may be formed or deposited in a confined area or areas of the skin-facing side of the backing material 602. FIG. 6B further illustrates cutouts 616A, 616B (hereinafter collectively “cutouts 616”) that may be included in the second adhesive layer 606 around the patch electrodes 610. The cutouts 616 may reduce or eliminate interference of the second adhesive layer 606 with an electrical connection between the patch electrodes 610 of the arrhythmia attachment patch 600 and skin of the subject 202.

The electrode contacts 608 may include metal, metallization, electrically conductive ink, or other electrically conductive material(s) or structure(s) formed or deposited in or on the backing material 602. The electrode contacts 608 may be exposed or accessible at the device-facing side of the backing material 602. In addition, the electrode contacts 608 may be disposed at locations of the backing material 602 that at least partially align with the ECG device electrode locations 614. As such, the electrode contacts 608 may electrically couple the arrhythmia attachment patch 600 to the ECG device 300, and specifically to the electrodes 306 of the ECG device 300, when the arrhythmia attachment patch 600 is properly aligned with and coupled to the ECG device 300. In particular, when the ECG device 300 is coupled to the arrhythmia attachment patch 600 with the ECG device 300 aligned to the arrhythmia attachment patch 600 such that the electrodes 306 of the ECG device 300 are aligned to the ECG device electrode locations 614, the first electrode contact 608A may be electrically coupled to the first electrode 306A of the ECG device 300 and the second electrode contact 608B may be electrically coupled to the second electrode 306B of the ECG device 300.

A portion of the device-facing side of the backing material 602 to which the ECG device 300 is coupled, generally corresponding to the first adhesive layer 604 in this example, together with the electrode contacts 608 and/or the first adhesive layer 604, may define a common patch electromechanical interface 618. The common patch electromechanical interface 618 may include the portion of the device-facing side of the backing material 602, the electrode contacts 608 positioned to align with and couple to the electrodes 306 of the ECG device 300, and/or the first adhesive layer 604. The common patch electromechanical interface 618 may be configured to electromechanically couple the arrhythmia attachment patch 600 to ECG devices, such as the ECG device 300. For example, the portion of the device-facing side of the backing material 602, together with the first adhesive layer 604, may mechanically couple the arrhythmia attachment patch 600 to the ECG device 300 and the electrode contacts 608 may electrically couple the arrhythmia attachment patch 600 to the electrodes 306 of the ECG device 300.

The common patch electromechanical interface 618 of the arrhythmia attachment patch 600 may be the same as the common patch electromechanical interface 418 of the non-arrythmia attachment patch 400, e.g., same dimensions, same arrangement, etc. As such, the common patch electromechanical interface 618 of the arrhythmia attachment patch 600 may electromechanically couple the arrhythmia attachment patch 600 to the same ECG devices as the common patch electromechanical interface 418 of the non-arrythmia attachment patch 400.

The patch electrodes 610 may include metal, metallization, electrically conductive ink, or other electrically conductive material(s) or structure(s) formed or deposited in or on the backing material 602. The patch electrodes 610 may be exposed or accessible at the skin-facing side of the backing material 602. In addition, the patch electrodes 610 may have a spacing according to a desired use of the arrhythmia attachment patch 600. For example, in some embodiments, the patch electrodes 610 may be space about 85 mm apart, or other distance. The spacing of the patch electrodes 610 may refer to a center-to-center spacing of the patch electrodes 610.

The patch electrodes 610 may be electrically coupled to the electrode contacts 608. In the illustrated embodiment, the arrhythmia attachment patch 600 further includes a first electrically conductive structure 620A to electrically couple the first patch electrode 610A to the first electrode contact 608A and a second electrically conductive structure 620B to electrically couple the second patch electrode 610B to the second electrode contact 608B. The first and second electrically conductive structures 620A, 620B are referred to hereinafter collectively as “electrically conductive structures 620” or generically as “electrically conductive structure 620”. Each of the electrically conductive structures 620 may include one or more electrical traces, one or more wires, one or more nanowires, one or more electrically conductive ink structures, or other suitable electrically conductive structure to electrically couple a corresponding one of the patch electrodes 610 to a corresponding one of the electrode contacts 608. Alternatively or additionally, each of the electrically conductive structures 620 may include metal, metallization, electrically conductive ink, or other electrically conductive material(s) or structure(s).

FIG. 7 includes a cross-sectional view of an ECG system 700 that includes the ECG device 300 of FIGS. 3A-3C and the arrhythmia attachment patch 600 of FIGS. 6A-6C, arranged in accordance with at least one embodiment described herein. The cross-sectional view of FIG. 7 is taken from the same direction as the cross-sectional view of FIG. 6C with the addition of the ECG device 300 electromechanically coupled to the arrhythmia attachment patch 600.

As illustrated in FIG. 7, the ECG device electromechanical interface 308 and the common patch electromechanical interface 618 cooperate to electromechanically couple the ECG device 300 to the arrhythmia attachment patch 600. In particular, the electrodes 306 of the ECG device 300 and the electrode contacts 608 of the arrhythmia attachment patch 600 cooperate to electrically couple the ECG device 300 to the arrhythmia attachment patch 600 while the bottom surface of the housing 302 of the ECG device 300, the portion of the device-facing side of the backing material 602, and the first adhesive layer 604 cooperate to mechanically couple the ECG device 300 to the arrhythmia attachment patch 600.

FIG. 7 further illustrates the ECG system 700 coupled to skin 702 of a subject, such as the subject 202 of FIG. 2A. In particular, the second adhesive layer 606 may mechanically couple the ECG system 700 to the skin 702. In some embodiments, a hydrogel 704 or other electrically conductive substance may be placed on the patch electrodes 610, e.g., within the cutouts 616 (FIGS. 6B-6C), before the ECG system 700 is coupled to the skin 702 to electrically couple the skin 702 to the patch electrodes 610, and more generally to the ECG system 700, when the ECG system 700 is mechanically coupled to the skin 702.

Some embodiments herein include an ECG device, such as the ECG device 212, 300. Some embodiments herein include an ECG system that includes both an ECG device and a first type of attachment patch, such as a non-arrythmia attachment patch. Some embodiments herein include an ECG system that includes both an ECG device and a second type of attachment patch, such as an arrhythmia attachment patch. Some embodiments herein include an ECG system that includes an ECG device, a first type of attachment patch, and a second type of attachment patch, or even more types of attachment patches.

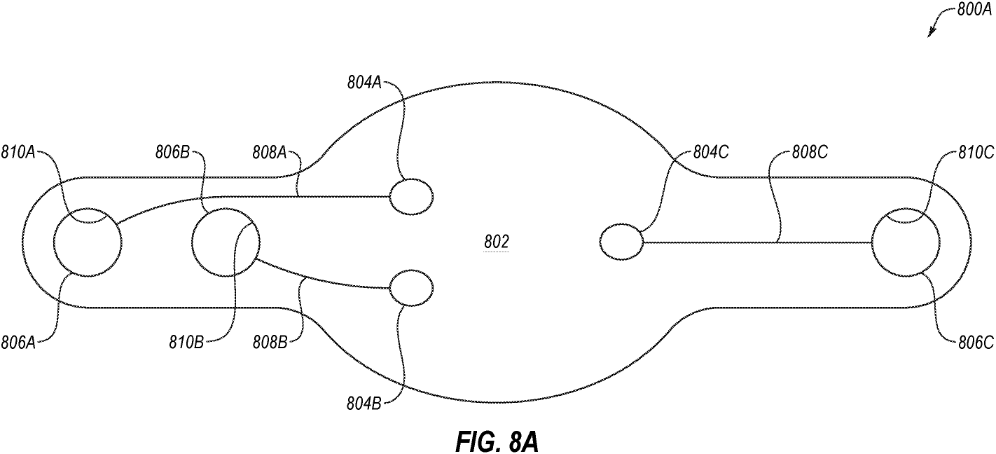

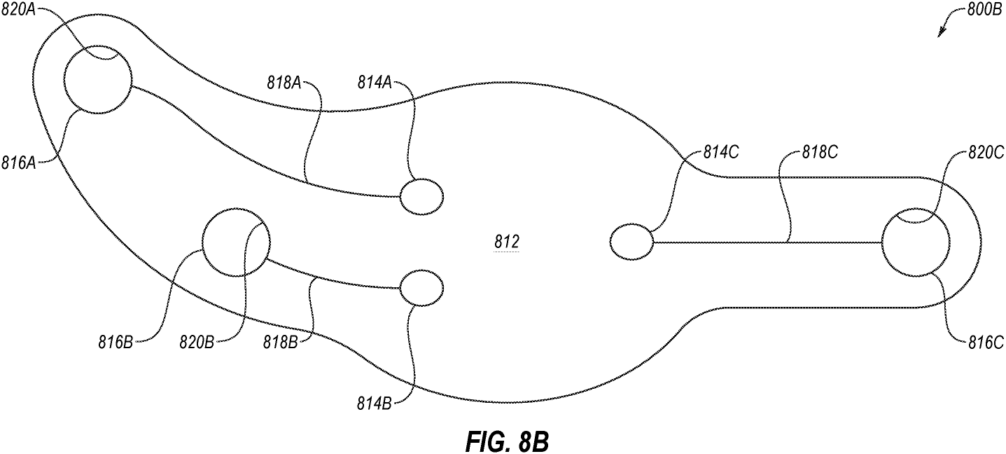

FIG. 8A is a bottom view of another attachment patch 800A that may be implemented in an ECG system, arranged in accordance with at least one embodiment described herein. The attachment patch 800A may include, be included in, or correspond to other attachment patches described herein. In general, the attachment patch 800A may be configured to couple an ECG device with more than two electrodes, specifically three electrodes, to a subject, such as the subject 202 of FIGS. 2A or 2B.

In some embodiments, the attachment patch 800A may include a backing material or substrate (not shown in FIG. 8A) with a first adhesive layer (not shown in FIG. 8A) formed or deposited on a device-facing side thereof and a second adhesive layer 802 formed or deposited on an opposite or skin-facing side thereof. The backing material may include any suitable backing material. The first adhesive layer and the second adhesive layer 802 may include any suitable adhesive.

The attachment patch 800A may additionally include electrode contacts 804A, 804B, 804C (hereinafter collectively “electrode contacts 804” or generically “electrode contact 804”) and patch electrodes 806A, 806B, 806C (hereinafter collectively “patch electrodes 806” or generically “patch electrode 806”). The patch electrodes 806 may be electrically coupled to the electrode contacts 804 via electrically conductive structures 808A, 808B, 808C (hereinafter collectively “electrically conductive structures 808”), such as one or more electrical traces, one or more wires, one or more nanowires, one or more conductive ink structures, or the like. Specifically, the electrically conductive structure 808A electrically couples the patch electrode 806A to the electrode contact 804A, the electrically conductive structure 808B electrically couples the patch electrode 806B to the electrode contact 804B, and the electrically conductive structure 808C electrically couples the patch electrode 806C to the electrode contact 804C.

The attachment patch 800A may generally be configured in the same or similar manner as other attachment patches described herein, e.g., with a common patch electromechanical interface (not shown in FIG. 8A) that is complementary to an ECG device electromechanical interface that includes three electrodes, the electrode contacts 804 exposed at the device-facing side to electrically couple to corresponding electrodes of the ECG device, the patch electrodes 806 exposed at the skin-facing side to electrically couple to the subject, the first adhesive layer formed on all or a portion of the device-facing side of the backing material, the second adhesive layer 802 formed on all or a portion of the skin-facing side, cutouts formed in the first adhesive layer for the electrode contacts 804, cutouts 810A, 810B, 810C formed in the second adhesive layer 802 for the patch electrodes 806, etc.

FIG. 8B is a bottom view of another attachment patch 800B that may be implemented in an ECG system, arranged in accordance with at least one embodiment described herein. The attachment patch 800B may include, be included in, or correspond to other attachment patches described herein. In general, the attachment patch 800B may be configured to couple an ECG device with more than two electrodes, specifically three electrodes, to a subject, such as the subject 202 of FIGS. 2A or 2B. The attachment patch 800B may have the same common patch electromechanical interface as the attachment patch 800A such that both the attachment patches may be electromechanically coupled to the same ECG device having three electrodes in the ECG device electromechanical interface.

In some embodiments, the attachment patch 800B may include a backing material or substrate (not shown in FIG. 8B) with a first adhesive layer (not shown in FIG. 8B) formed or deposited on a device-facing side thereof and a second adhesive layer 812 formed or deposited on an opposite or skin-facing side thereof. The backing material may include any suitable backing material. The first adhesive layer and the second adhesive layer 812 may include any suitable adhesive.

The attachment patch 800B may additionally include electrode contacts 814A, 814B, 814C (hereinafter collectively “electrode contacts 814” or generically “electrode contact 814”) and patch electrodes 816A, 816B, 816C (hereinafter collectively “patch electrodes 816” or generically “patch electrode 816”). The patch electrodes 816 may be electrically coupled to the electrode contacts 814 via electrically conductive structures 818A, 818B, 818C (hereinafter collectively “electrically conductive structures 818”), such as one or more electrical traces, one or more wires, one or more nanowires, one or more conductive ink structures, or the like. Specifically, the electrically conductive structure 818A electrically couples the patch electrode 816A to the electrode contact 814A, the electrically conductive structure 818B electrically couples the patch electrode 816B to the electrode contact 814B, and the electrically conductive structure 818C electrically couples the patch electrode 816C to the electrode contact 814C.

The attachment patch 800B may generally be configured in the same or similar manner as other attachment patches described herein, e.g., with a common patch electromechanical interface (not shown in FIG. 8B) that is complementary to an ECG device electromechanical interface that includes three electrodes, the electrode contacts 814 exposed at the device-facing side to electrically couple to corresponding electrodes of the ECG device, the patch electrodes 816 exposed at the skin-facing side to electrically couple to the subject, the first adhesive layer formed on all or a portion of the device-facing side of the backing material, the second adhesive layer 812 formed on all or a portion of the skin-facing side, cutouts formed in the first adhesive layer for the electrode contacts 814, cutouts 820A, 820B, 820C formed in the second adhesive layer 812 for the patch electrodes 816, etc.

The attachment patches 800A, 800B may have the common patch electromechanical interface that allows the attachment patches 800A, 800B to be electromechanically coupled to identical ECG devices, e.g., different instances of the same ECG sensor platform, and specifically an ECG sensor platform with three electrodes. In comparison, the attachment patches 400, 600 have the common patch electromechanical interface 418, 618 that allows the attachment patches 400, 600 to be electromechanically coupled to identical ECG devices of a different ECG sensor platform, and specifically the ECG platform depicted in FIGS. 3A-3C with two electrodes. More generally, attachment patches herein that share a common patch electromechanical interface may be electromechanically coupled to identical ECG devices with any number of electrodes (e.g., two, three, four, five, etc.). In these and other embodiments, the attachment patches may have a corresponding number (e.g., two, three, four, five, etc.) of electrode contacts and a corresponding number (e.g., two, three, four, five, etc.) of patch electrodes. Moreover, different types of attachment patches having a given number of patch electrodes may have the patch electrodes arranged in different patterns or arrangements according to a desired application or implementation.

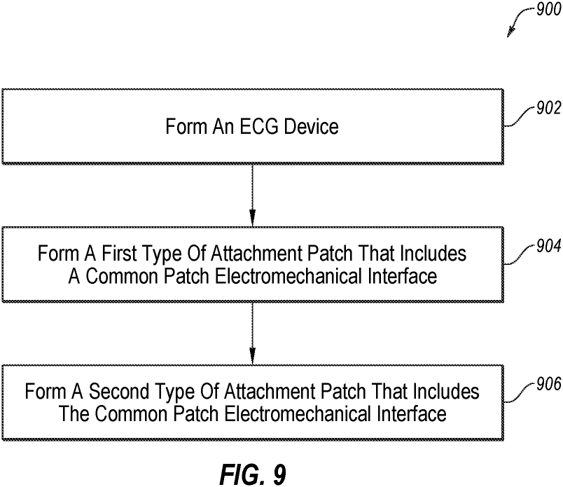

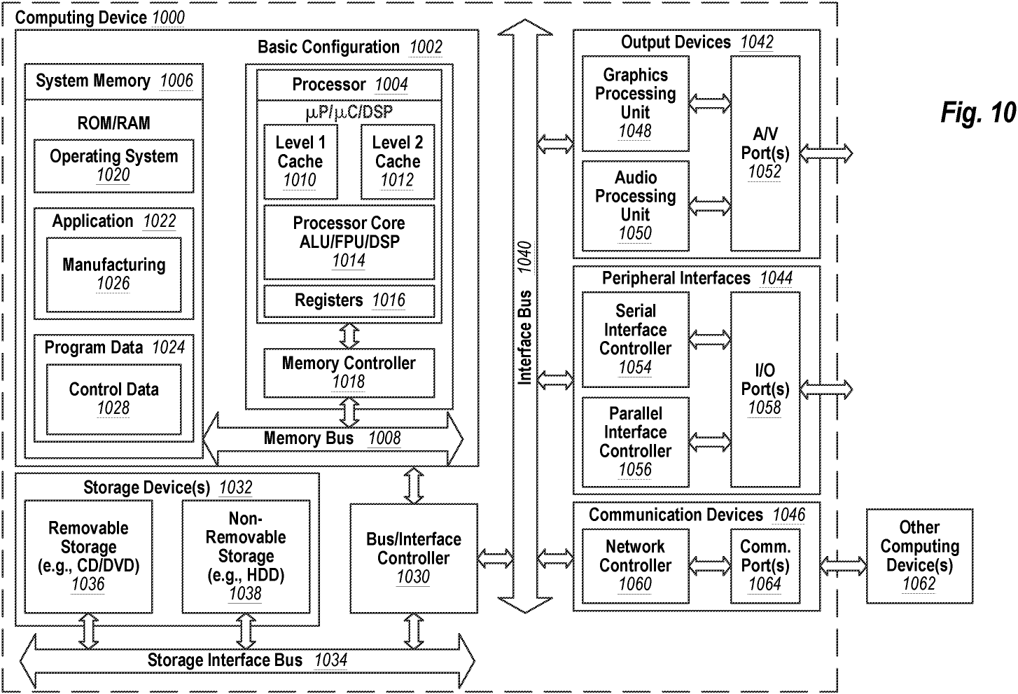

FIG. 9 is a flowchart of a manufacturing method 900, arranged in accordance with at least one embodiment described herein. The method 900 may be programmably performed or controlled by one or more processors, in, e.g., one or more computing devices. In an example implementation, the method 900 may be performed and/or controlled in whole or in part by a computing device 1000 depicted in FIG. 10. The method 900 may include one or more of blocks 902, 904, and/or 906.

At block 902, the method 900 may including forming an ECG device that includes a housing, an ECG sensor disposed in the housing, and first and second electrodes accessible from outside the housing and electrically coupled to the ECG sensor. For example, block 902 may include forming the ECG device 212, 300 of FIGS. 2A-3B. The housing and the first and second electrodes may define an ECG device electromechanical interface, such as the ECG device electromechanical interface 308 of the ECG device 300.

At block 904, the method 900 may include forming a first type of attachment patch that includes a common patch electromechanical interface that is complementary to the ECG device electromechanical interface and two first patch electrodes with a first spacing. For example, block 904 may include forming the first type of attachment patch 214A of FIG. 2A and/or the non-arrythmia attachment patch 400 of FIGS. 4A-4C. In some embodiments, forming the first type of attachment patch that includes the two first patch electrodes with the first spacing at block 904 may include forming the two first patch electrodes exposed on a skin-facing side of the first type of attachment patch and electrically coupled to two electrode contacts on a device-facing side of the first type of attachment patch, the two electrode contacts configured to align with and electrically couple to the first and second electrodes of the ECG device. The two first patch electrodes may include the patch electrodes 410 of the non-arrythmia attachment patch 400. The two electrode contacts may include the electrode contacts 408 of the non-arrythmia attachment patch 400.

At block 906, the method 900 may include forming a second type of attachment patch that includes the common patch electromechanical interface that is complementary to the ECG device electromechanical interface and two second patch electrodes with a second spacing that is greater than the first spacing. For example, block 906 may include forming the second type of attachment patch 214B of FIG. 2B and/or the arrhythmia attachment patch 600 of FIGS. 6A-6C. In some embodiments, forming the second type of attachment patch that includes two second patch electrodes with the second spacing at block 906 may include forming first and second electrode contacts in the second type of attachment patch that are exposed at a device-facing side of the second type of attachment patch and that are configured to align with and electrically couple to the first and second electrodes of the ECG device. The first and second electrode contacts may include the electrode contacts 608 of the arrhythmia attachment patch. Block 906 may further include forming first and second patch electrodes with the second spacing in the second type of attachment patch that are exposed at a skin-facing side of the second type of attachment patch opposite the device-facing surface. The first and second patch electrodes may include the patch electrodes 610 of the arrhythmia attachment device 600. Block 906 may further include forming a first electrically conductive structure in the second type of attachment patch that electrically couples the first electrode contact to the first patch electrode and forming a second electrically conductive structure in the second type of attachment patch that electrically couples the second electrode contact to the second patch electrode. The first and second electrically conductive structures may include the electrically conductive structures 620 of the arrhythmia attachment patch. Forming each of the first and second electrically conductive structures may include forming one or more electrical traces, one or more wires, one or more nanowires, or one or more electrically conductive ink structures in the second type of attachment patch.

One skilled in the art will appreciate that, for this and other processes and methods disclosed herein, the functions performed in the processes and methods may be implemented in differing order. Further, the outlined steps and operations are only provided as examples, and some of the steps and operations may be optional, combined into fewer steps and operations, or expanded into additional steps and operations without detracting from the essence of the disclosed embodiments.

In some embodiments, forming the first type of attachment patch that includes two first patch electrodes with the first spacing at block 904 may include forming the two first patch electrodes in the first type of attachment patch with the first spacing of about 35 millimeters, and forming the second type of attachment patch that includes two second patch electrodes with the second spacing at block 906 may include forming the two second patch electrodes in the second type of attachment patch with the second spacing of about 85 millimeters.

In some embodiments, the ECG device electromechanical interface of the ECG device and the common patch electromechanical interface of each of the first and second type of attachment patch are configured to cooperate to electromechanically couple the ECG device to the first type of attachment patch and the second type of attachment patch.

In some embodiments, each of the first and second type of attachment patch is configured to be electrically coupled to the ECG device through the common patch electromechanical interface and the ECG device electromechanical interface and to skin of a subject through the two first or two second patch electrodes. The first type of attachment patch may include a non-arrythmia attachment patch, such as the non-arrythmia attachment patch 214A or 400. The second type of attachment patch may include an arrythmia attachment patch, such as the arrhythmia attachment patch 214B or 600. Each non-arrythmia attachment patch may be configured to direct electrical signals from locations of the subject at which the two first patch electrodes are positioned when the non-arrythmia attachment patch is coupled to the skin of the subject to, respectively, the first and second electrodes of the ECG device. Each arrythmia attachment patch may be configured to direct electrical signals from locations of the subject at which the two second patch electrodes are positioned when the arrythmia attachment patch is coupled to the skin of the subject to, respectively, the first and second electrodes of the ECG device.

FIG. 10 is a block diagram illustrating an example computing device 1000, arranged in accordance with at least one embodiment described herein. The computing device 1000 may include, be included in, or otherwise correspond to, e.g., the personal electronic devices 204, the server 206, the ECG device 212, 300, or a computing device in a factory that performs or controls performance of, e.g., the manufacturing method 900 of FIG. 9. In a basic configuration 1002, the computing device 1000 typically includes one or more processors 1004 and a system memory 1006. A memory bus 1008 may be used to communicate between the processor 1004 and the system memory 1006.

Depending on the desired configuration, the processor 1004 may be of any type including, but not limited to, a microprocessor (µP), a microcontroller (µC), a digital signal processor (DSP), or any combination thereof. The processor 1004 may include one or more levels of caching, such as a level one cache 1010 and a level two cache 1012, a processor core 1014, and registers 1016. The processor core 1014 may include an arithmetic logic unit (ALU), a floating point unit (FPU), a digital signal processing core (DSP Core), or any combination thereof. An example memory controller 1018 may also be used with the processor 1004, or in some implementations the memory controller 1018 may include an internal part of the processor 1004.

Depending on the desired configuration, the system memory 1006 may be of any type including volatile memory (such as RAM), nonvolatile memory (such as ROM, flash memory, etc.), or any combination thereof. The system memory 1006 may include an operating system 1020, one or more applications 1022, and program data 1024. The application 1022 may include a manufacturing application 1026 that is arranged to perform or control performance of a manufacturing method. The program data 1024 may include manufacturing control data 1028 to control the manufacturing method. In some embodiments, the application 1022 may be arranged to operate with the program data 1024 on the operating system 1020 such that one or more methods may be provided as described herein, including the method 900 of FIG. 9.

The computing device 1000 may have additional features or functionality, and additional interfaces to facilitate communications between the basic configuration 1002 and any involved devices and interfaces. For example, a bus/interface controller 1030 may be used to facilitate communications between the basic configuration 1002 and one or more data storage devices 1032 via a storage interface bus 1034. The data storage devices 1032 may be removable storage devices 1036, non-removable storage devices 1038, or a combination thereof. Examples of removable storage and non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

The system memory 1006, the removable storage devices 1036, and the non-removable storage devices 1038 are examples of computer storage media or non-transitory computer-readable media. Computer storage media or non-transitory computer-readable media includes RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other non-transitory medium which may be used to store the desired information and which may be accessed by the computing device 1000. Any such computer storage media or non-transitory computer-readable media may be part of the computing device 1000.

The computing device 1000 may also include an interface bus 1040 to facilitate communication from various interface devices (e.g., output devices 1042, peripheral interfaces 1044, and communication devices 1046) to the basic configuration 1002 via the bus/interface controller 1030. The output devices 1042 include a graphics processing unit 1048 and an audio processing unit 1050, which may be configured to communicate to various external devices such as a display or speakers via one or more A/V ports 1052. Diagrams, flowcharts, organizational charts, connectors, and/or other graphical objects generated by the diagram application 1026 may be output through the graphics processing unit 1048 to such a display. The peripheral interfaces 1044 include a serial interface controller 1054 or a parallel interface controller 1056, which may be configured to communicate with external devices such as input devices (e.g., keyboard, mouse, pen, voice input device, touch input device, etc.), sensors, or other peripheral devices (e.g., printer, scanner, etc.) via one or more I/O ports 1058. Such input devices may be operated by a user to provide input to the diagram application 1026, which input may be effective to, e.g., generate curved connectors, designate points as designated points of one or more curved connectors, relocate one or more designated points, and/or to accomplish other operations within the diagram application 1026. The communication devices 1046 include a network controller 1060, which may be arranged to facilitate communications with one or more other computing devices 1062 over a network communication link via one or more communication ports 1064.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term “computer-readable media” as used herein may include both storage media and communication media.

The computing device 1000 may be implemented as a portion of a small-form factor portable (or mobile) electronic device such as a smartphone, a personal data assistant (PDA) or an application-specific device. The computing device 1000 may also be implemented as a personal computer including tablet computer, laptop computer, and/or non-laptop computer configurations, or a server computer including both rack-mounted server computer and blade server computer configurations.

Embodiments described herein may be implemented using computer-readable media for carrying or having computer-executable instructions or data structures stored thereon. Such computer-readable media may be any available media that may be accessed by a general-purpose or special-purpose computer. By way of example, such computer-readable media may include non-transitory computer-readable storage media including RAM, ROM, EEPROM, CD-ROM or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable media.

Computer-executable instructions may include, for example, instructions and data which cause a general-purpose computer, special-purpose computer, or special-purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless specific arrangements described herein are mutually exclusive with one another, the various implementations described herein can be combined to enhance system functionality or to produce complementary functions. Likewise, aspects of the implementations may be implemented in standalone arrangements. Thus, the above description has been given by way of example only and modification in detail may be made within the scope of the present invention.

With respect to the use of substantially any plural or singular terms herein, those having skill in the art can translate from the plural to the singular or from the singular to the plural as is appropriate to the context or application. The various singular/plural permutations may be expressly set forth herein for sake of clarity. A reference to an element in the singular is not intended to mean “one and only one” unless specifically stated, but rather “one or more.” Moreover, nothing disclosed herein is intended to be dedicated to the public regardless of whether such disclosure is explicitly recited in the above description.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that include A alone, B alone, C alone, A and B together, A and C together, B and C together, or A, B, and C together, etc.). Also, a phrase presenting two or more alternative terms, whether in the description, claims, or drawings, should be understood to include one of the terms, either of the terms, or both terms. For example, the phrase “A or B” will be understood to include the possibilities of “A” or “B” or “A and B.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An electrocardiogram (ECG) device, comprising:
a housing;
an ECG sensor disposed in the housing;
a first electrode accessible from outside the housing and electrically coupled to the ECG sensor; and
a second electrode accessible from outside the housing and electrically coupled to the ECG sensor;
wherein the housing and the first and second electrodes define an ECG device electromechanical interface that is complementary to a common patch electromechanical interface that is included in at least two different types of attachment patches.

2. The ECG device of claim 1, wherein:
the at least two different types of attachment patches include a first type and a second type; and
the ECG device electromechanical interface and the common patch electromechanical interface are configured to cooperate to electromechanically couple the ECG device to the first type of attachment patch and to the second type of attachment patch.

3. The ECG device of claim 1, wherein:
the at least two different types of attachment patches include non-arrythmia attachment patches and arrythmia attachment patches;
each non-arrythmia attachment patch is configured to direct electrical signals from locations of a subject spaced a first distance apart to, respectively, the first and second electrodes of the ECG device; and
each arrythmia attachment patch is configured to direct electrical signals from locations of the subject spaced a second distance apart to, respectively, the first and second electrodes of the ECG device, the first distance being less than the second distance.

4. A manufacturing method, comprising:
forming an electrocardiogram (ECG) device that includes a housing, an ECG sensor disposed in the housing, and first and second electrodes accessible from outside the housing and electrically coupled to the ECG sensor, wherein the housing and the first and second electrodes define an ECG device electromechanical interface;
forming a first type of attachment patch that includes a common patch electromechanical interface that is complementary to the ECG device electromechanical interface and two first patch electrodes with a first spacing; and
forming a second type of attachment patch that includes the common patch electromechanical interface that is complementary to the ECG device electromechanical interface and two second patch electrodes with a second spacing that is greater than the first spacing.

5. The manufacturing method of claim 4, wherein:
forming the first type of attachment patch that includes two first patch electrodes with the first spacing comprises forming the two first patch electrodes in the first type of attachment patch with the first spacing of about 35 millimeters; and
forming the second type of attachment patch that includes two second patch electrodes with the second spacing comprises forming the two second patch electrodes in the second type of attachment patch with the second spacing of about 85 millimeters.

6. The manufacturing method of claim 4, wherein forming the first type of attachment patch that includes the two first patch electrodes with the first spacing comprises forming the two first patch electrodes exposed on a skin-facing side of the first type of attachment patch and electrically coupled to two electrode contacts on a device-facing side of the first type of attachment patch, the two electrode contacts configured to align with and electrically couple to the first and second electrodes of the ECG device.

7. The manufacturing method of claim 4, wherein forming the second type of attachment patch that includes two second patch electrodes with the second spacing comprises:
forming first and second electrode contacts in the second type of attachment patch that are exposed at a device-facing side of the second type of attachment patch and that are configured to align with and electrically couple to the first and second electrodes of the ECG device;
forming first and second patch electrodes with the second spacing in the second type of attachment patch that are exposed at a skin-facing side of the second type of attachment patch opposite the device-facing surface;
forming a first electrically conductive structure in the second type of attachment patch that electrically couples the first electrode contact to the first patch electrode; and
forming a second electrically conductive structure in the second type of attachment patch that electrically couples the second electrode contact to the second patch electrode.

8. The manufacturing method of claim 7, wherein forming each of the first and second electrically conductive structures comprises forming one or more electrical traces, one or more wires, one or more nanowires, or one or more electrically conductive ink structures in the second type of attachment patch.

9. The manufacturing method of claim 4, wherein the ECG device electromechanical interface and the common patch electromechanical interface are configured to cooperate to electromechanically couple the ECG device to the first type of attachment patch and the second type of attachment patch.

10. The manufacturing method of claim 4, wherein:
each of the first and second type of attachment patch is configured to be electrically coupled to the ECG device through the common patch electromechanical interface and the ECG device electromechanical interface and to skin of a subject through the two first or two second patch electrodes;
the first type of attachment patch includes a non-arrythmia attachment patch;
the second type of attachment patch includes an arrythmia attachment patch;
each non-arrythmia attachment patch is configured to direct electrical signals from locations of the subject at which the two first patch electrodes are positioned when the non-arrythmia attachment patch is coupled to the skin of the subj ect to, respectively, the first and second electrodes of the ECG device; and
each arrythmia attachment patch is configured to direct electrical signals from locations of the subject at which the two second patch electrodes are positioned when the arrythmia attachment patch is coupled to the skin of the subject to, respectively, the first and second electrodes of the ECG device.

11. An electrocardiogram (ECG) system, comprising:
an ECG device that includes a housing, an ECG sensor disposed in the housing, and first and second electrodes accessible from outside the housing and electrically coupled to the ECG sensor, wherein the housing and the first and second electrodes define an ECG device electromechanical interface;
a first type of attachment patch that includes a common patch electromechanical interface that is complementary to the ECG device electromechanical interface and two first patch electrodes with a first spacing; and
a second type of attachment patch that includes the common patch electromechanical interface that is complementary to the ECG device electromechanical interface and two second patch electrodes with a second spacing that is greater than the first spacing.

12. The ECG system of claim 11, wherein:
the first spacing of the two first patch electrodes of the first type of attachment patch is about 35 millimeters; and
the second spacing of the two second patch electrodes of the second type of attachment patch is about 85 millimeters.

13. The ECG system of claim 11, wherein the two first patch electrodes are disposed on a skin-facing side of the first type of attachment patch and coupled to two electrode contacts disposed on a device-facing side of the first type of attachment patch, the two electrode contacts configured to align with the first and second electrodes of the ECG device.

14. The ECG system of claim 11, wherein:
the second type of attachment patch further comprises first and second electrode contacts in the second type of attachment patch that are exposed at a device-facing side of the second type of attachment patch and that are configured to align with and electrically couple to the first and second electrodes of the ECG device;
the two second patch electrodes comprise first and second patch electrodes with the second spacing in the second type of attachment patch that are exposed at a skin-facing side of the second type of attachment patch opposite the device-facing side; and
the second type of attachment patch further comprises:
a first electrically conductive structure in the second type of attachment patch that electrically couples the first electrode contact to the first patch electrode; and
a second electrically conductive structure in the second type of attachment patch that electrically couples the second electrode contact to the second patch electrode.

15. The ECG system of claim 14, wherein each of the first and second electrically conductive structures comprises one or more electrical traces, one or more wires, one or more nanowires, or one or more electrically conductive ink structures formed in the second type of attachment patch.

16. The ECG system of claim 11, wherein the ECG device electromechanical interface and the common patch electromechanical interface are configured to cooperate to electromechanically couple the ECG device to an attachment patch of the first type and an attachment patch of the second type.

17. The ECG system of claim 11, wherein:
each of the first and second type of attachment patch is configured to be electrically coupled to the ECG device through the common patch electromechanical interface and the ECG device electromechanical interface and to skin of a subject through the two first or two second patch electrodes;
the first type of attachment patch includes a non-arrythmia attachment patch;
the second type of attachment patch includes an arrythmia attachment patch;
each non-arrythmia attachment patch is configured to direct electrical signals from locations of the subject at which the two first patch electrodes are positioned when the non-arrythmia attachment patch is coupled to the skin of the subject to, respectively, the first and second electrodes of the ECG device; and
each arrythmia attachment patch is configured to direct electrical signals from locations of the subject at which the two second patch electrodes are positioned when the arrythmia attachment patch is coupled to the skin of the subject to, respectively, the first and second electrodes of the ECG device.

18. An electrocardiogram (ECG) system, comprising:
an electrocardiogram (ECG) device that includes a housing, an ECG sensor disposed in the housing, and first and second electrodes accessible from outside the housing and electrically coupled to the ECG sensor, wherein the housing and the first and second electrodes define an ECG device electromechanical interface; and
a non-arrythmia attachment patch that includes a common patch electromechanical interface that is complementary to the ECG device electromechanical interface and two patch electrodes with a predetermined spacing;
wherein:
the non-arrythmia attachment patch is configured to be electrically coupled to the ECG device through the common patch electromechanical interface and the ECG device electromechanical interface and to skin of a subject through the two patch electrodes; and
the non-arrythmia attachment patch is configured to direct electrical signals from locations of the subject at which the two patch electrodes are positioned when the non-arrythmia attachment patch is coupled to the skin of the subject and spaced apart by the predetermined spacing to, respectively, the first and second electrodes of the ECG device.

19. The ECG system of claim 18, wherein the ECG sensor is configured to measure timing between successive R-R waveforms of the subject.

20. An electrocardiogram (ECG) system, comprising:
an electrocardiogram (ECG) device that includes a housing, an ECG sensor disposed in the housing, and first and second electrodes accessible from outside the housing and electrically coupled to the ECG sensor, wherein the housing and the first and second electrodes define an ECG device electromechanical interface; and
an arrythmia attachment patch that includes a common patch electromechanical interface that is complementary to the ECG device electromechanical interface and two patch electrodes with a predetermined spacing;
wherein:
the arrythmia attachment patch is configured to be electrically coupled to the ECG device through the common patch electromechanical interface and the ECG device electromechanical interface and to skin of a subject through the two patch electrodes; and
the arrythmia attachment patch is configured to direct electrical signals from locations of the subject at which the two patch electrodes are positioned when the arrythmia attachment patch is coupled to the skin of the subject and spaced apart by the predetermined spacing to, respectively, the first and second electrodes of the ECG device.

21. The ECG system of claim 20, wherein the ECG sensor is configured to measure a PQRS waveform of the subject.
